(19) United States
(12) Patent Application Publication
(10) Pub. No.: US 2019/0167727 A1
(43) Pub. Date: Jun. 6, 2019

(54) NEUROPROTECTIVE COMPOSITION, PREPARATION PROCESS THEREOF AND MEDICAL USES THEREOF

(71) Applicant: Caire Medical-Biotechnology International Co., Taipei City (TW)

(72) Inventors: Te-Fu CHEN, Taipei City (TW); Kuo-Chuan WANG, Taipei City (TW)

(21) Appl. No.: 16/106,290

(22) Filed: Aug. 21, 2018

Related U.S. Application Data
(62) Division of application No. 15/830,200, filed on Dec. 4, 2017.

(51) Int. Cl. A61K 35/28; A61P 9/10; A61P 37/06; A61P 25/26; A61P 25/16; C12N 5/0775

(52) U.S. Cl. CPC A61K 35/28; A61P 9/10; A61P 37/06; A61P 25/26; C12N 2502/1364; A61P 25/16; C12N 5/0664

(57) ABSTRACT

The invention relates to a neuroprotective composition derived from mesenchymal stem cells, especially a neuroprotective composition derived from the primary culture of dental pulp mesenchymal stem cells. The invention also relates to a process for preparing the neuroprotective composition, as well as the medical use of the neuroprotective composition in the treatment of Parkinson's disease.

### FIELD OF THE INVENTION

The invention relates to a neuroprotective composition derived from mesenchymal stem cells, especially a neuroprotective composition derived from the primary culture of dental pulp mesenchymal stem cells, its preparation process and its medical uses in the treatment of neurological diseases associated with neuronal damage, including subarachnoid hemorrhage and Parkinson's disease.

### DESCRIPTION OF THE RELATED ART

Subarachnoid hemorrhage (SAH) is extravasation of blood into the subarachnoid space between the arachnoid membrane and the pia mater surrounding the brain. It occurs in various clinical contexts, the most common being head trauma, while nontraumatic (or spontaneous) subarachnoid hemorrhage usually occurs in the setting of a ruptured cerebral aneurysm or arteriovenous malformation. Risk factors for spontaneous cases included high blood pressure, smoking, family history, alcoholism, and cocaine use.

Aneurysmal SAH carries significant morbidity and mortality. Nearly half of people with aneurysmal SAH die within 30 days, with one-third of survivors suffering long-term physical, neurocognitive, psychiatric, and/or psychological symptoms, such as hemiplegia, mood disorders, frequent headaches, cognitive and memory impairment. SAH patients could also have an increased likelihood of suffering from Alzheimer's disease or dementia in old age. In addition to the original bleeding, factors such as hypoxia, hypotension, cerebral edema, re-bleeding, delayed ischemic neurological deficits (DINDs), and/or ischemia due to cerebral arterial manipulation during clipping or coiling all contribute to secondary brain injury. If not given a correct medical treatment in time, the patients survived for the first bleeding may occur with bleeding again within 3 weeks, the mortality of which is as high as 80%.

Once red blood cells run into subarachnoid area and lysis of the red blood cells occurs, it will induce chemical meningitis which in turn causes a series of complex pathophysiological process, including the intracranial pressure increasing, cerebral blood flow and cerebral perfusion pressure reducing, blood-brain barrier (BBB) damage, brain edema, acute cerebral vasospasm, microvascular dysfunction and neuron apoptosis mechanism. The above process may secondarily cause calcium overload, free radicals accumulation, mitochondrial dysfunction, and immune inflammatory reaction. While cerebral vasospasm is considered the most common cause of disability and mortality among survivors of SAH, other influences such as early brain injury due to cortical spreading depression, disruption of the blood-brain barrier, impaired function of the microcirculation, neuroinflammation, and apoptotic neuronal cell death may contribute to SAH-induced pathologies. It is known in the art that the pathogenesis of SAH shares some common pathological features with other neurological disorders and neurological injury.

Despite the severity of SAH and the complications associated therewith, current measures for the treatment of SAH are relatively ineffective. Therefore, there exists a need for a therapeutic composition which provides protection to nervous tissue.

### SUMMARY OF THE INVENTION

The paracrine effects of stem cells on neurological disorders have been noticed for decades (see, for example, Torrente D. et al., Hum Exp Toxicol. 2014, 33(7): 673-84; and, for review, Martinez-Garza D. M. et al., Medicina Universitaria 2016, 18(72):169-180; Im W. S. and Kim M. H., J. Mov. Disord. 2014, 7(1):1-6; Turgeman G., Neural. Regen. Res. 2015 May, 10(5): 698-699; and Hasan A. et al., Front. Neurol. 8: 28, 2017, DOI: 10.3389/fneur.2017.00028). It was hypothesized that stem cells may secrete a variety of growth factors, cytokines, and chemokines that may enhance cell survival, increase neurogenesis, reduce inflammation and mitochondrial function, and all of these effects result in neural protection and repair. As such, introduction of stem cell secretome instead of whole stem cells into damaged tissues was considered as a promising and safety therapeutic measure to overcome the limitations of cell-based transplantation. While some paracrine molecules released by stem cells have been identified, including Scurfin, brain-derived neurotrophic factor (BDNF) and CC chemokine ligand 2 (CCL2), all of the proteins identified have molecular weights of more than 10 kDa. Surprisingly and unexpectedly, the present inventors found that the □5 kDa fraction of the conditioned medium derived from the primary culture of mesenchymal stem cells (MSCs), such as that derived from the primary culture of dental pulp mesenchymal stem cells (DPMSCs), exhibits excellent neuroprotective activity. The medium fraction is evidently useful for the therapeutic treatment of neurological disorders, such as SAH and Parkinson's disease.

Accordingly, in the first aspect provided herein is a neuroprotective composition obtainable by a process comprising the steps of:
(i) culturing mesenchymal stem cells in a serum-free basal culture medium for at least 3 hours to obtain a cell culture; and
(ii) processing the cell culture obtained in step (i) to obtain an aqueous fraction with a molecular weight of no more than about 5 kDa as the neuroprotective composition.

In the second aspect provided herein is a process of preparing a neuroprotective composition, comprising the steps of:
(i) culturing mesenchymal stem cells in a serum-free basal culture medium for at least 3 hours to obtain a cell culture; and
(ii) processing the cell culture obtained in step (i) to obtain an aqueous fraction with a molecular weight of no more than about 5 kDa as the neuroprotective composition.

In the third aspect provided herein is a neuroprotective composition for use in treatment of a neurological disorder associated with neuronal damage in a subject, wherein the neuroprotective composition is obtainable by the process described above.

In the fourth aspect provided herein is a method for treating a neurological disorder associated with neuronal damage in a subject, comprising administering to said subject an effective amount of a neuroprotective composition to suppress neuronal damage; wherein the neuroprotective composition is obtainable by the process described above.

In the fifth aspect provided herein is a neuroprotective composition for use in protecting against neuronal damage in a subject having or at risk of having loss of neural function, wherein the neuroprotective composition is obtainable by the process described above.

In the sixth aspect provided herein is a method of protecting against neuronal damage, comprising administering to a subject having or at risk of having loss of neural function an effective amount of a neuroprotective composition, thereby protecting against neuronal damage in the subject; wherein the neuroprotective composition is obtainable by the process described above.

In the seventh aspect provided herein is a neuroprotective composition for use in inhibiting cerebral neuroinflammation in a subject in need thereof, wherein the neuroprotective composition is obtainable by the process described above.

In the eighth aspect provided herein is a method of inhibiting cerebral neuroinflammation in a subject in need thereof, comprising administering an effective amount of a neuroprotective composition, thereby inhibiting cerebral neuroinflammation in the subject; wherein the neuroprotective composition is obtainable by the process described above.

In the preferred embodiments, the processing step (ii) comprises ultrafiltrating the cell culture obtained in step (i) through a membrane having a molecular weight cut-off of 5 kDa, thereby collecting a filtrate passing through the membrane as the neuroprotective composition.

In the preferred embodiments, the mesenchymal stem cells are dental pulp mesenchymal stem cells.

In a preferred embodiment, the neurological disorders associated with neuronal damage is selected from the group consisting of amyotrophic lateral sclerosis, Alzheimer's disease, Parkinson's disease, Huntington's disease, muscular dystrophy, multiple sclerosis, ischemic stroke, hemorrhagic stroke, transient ischemic attack (TIA), and traumatic brain injury (TBI). In a more preferred embodiment, the neurological disorder associated with neuronal damage is selected from the group consisting of Alzheimer's disease, Parkinson's disease, Huntington's disease, ischemic stroke, primary SAH, secondary SAH, traumatic SAH and intracerebral hemorrhage (ICH), transient ischemic attack (TIA), and traumatic brain injury (TBI). In an even more preferred embodiment, the neurological disorder associated with neuronal damage is selected from the group consisting of Parkinson's disease, primary SAH and secondary SAH.

### BRIEF DESCRIPTION OF THE DRAWINGS

The above and other objects, features and effects of the invention will become apparent with reference to the following description of the preferred embodiments taken in conjunction with the accompanying drawings, in which:

FIGS. 1A-1C are images showing the microcirculation vasculature on the rat brain surfaces in the Control group (FIG. 1A), the SAH group (FIG. 1B) and the Treated group (FIG. 1C), where the arterioles and venules in rat brains were marked by the letters A and V, respectively;

FIGS. 2A and 2B are histograms showing regional blood flow and brain tissue oxygen pressure at the brain surfaces in the SAH rat model;

FIG. 2C is histological images showing the Ibal-positive microglial cells in the brain tissues of the SAH rat model;

FIG. 3 is a histogram showing the enhancing effect of the present neuroprotective composition on neuron cell viability;

FIGS. 4A and 4B are histograms showing regional blood flow and brain tissue oxygen pressure at the brain surfaces in the D-gal-induced rat hepatic encephalopathy model;

FIG. 4C is histological images showing the TUNEL-positive glia cells in the brain tissues of the D-gal-induced rat hepatic encephalopathy model;

FIGS. 5A and 5B are fluorescence microscopic images of the zebrafish treated with the present neuroprotective composition; and

FIG. 6 is a histogram showing the effects of the medium fraction disclosed herein on rotarod activity in rotenone-lesioned rat models, where rats in the respective groups were subjected to rotarod test before the rotenone treatment (pre-test), after the rotenone treatment (lesion) and after the administration of the medium fraction.

### DETAILED DESCRIPTION OF THE INVENTION

Unless specified otherwise, the following terms as used in the specification and appended claims are given the following definitions. It should be noted that the indefinite article “a” or “an” as used in the specification and claims is intended to mean one or more than one, such as “at least one,” “at least two,” or “at least three,” and does not merely refer to a singular one. In addition, the terms “comprising/comprises,” “including/includes” and “having/has” as used in the claims are open languages and do not exclude unrecited elements. The term “or” generally covers “and/or”, unless otherwise specified. The term “about” used throughout the specification and appended claims is used to describe and account for slight changes that do not materially affect the nature of the invention.

The present invention is based on the discovery that the ≤5 kDa aqueous fraction derived from the conditioned medium of mesenchymal stem cells (MSCs), especially that derived from dental pulp mesenchymal stem cells (DPMSCs), possesses neuroprotective activity, enhances neuronal survival, improves cerebral microcirculation, reduces neuroinflammation and alleviates vasoconstriction, indicating that the medium fraction is therapeutically effective in the treatment of a neurological disorder associated with neuronal damage.

The term “mesenchymal stem cells” or abbreviated as “MSCs” is used herein to refer to multipotent stem cells derived from adult stromal tissues, including but not limited to bone marrow, adipose tissue, muscle tissue, dental pulp, umbilical cord blood, amniotic fluid, skeletal muscle, synovial and Wharton's jelly, and having the property of extensive self-renewal and the ability to differentiate into cells of mesenchymal lineages. In the preferred embodiments, the MSCs used herein are derived from dental pulp tissues. The MSCs used in the invention may be collected from human, rat, mouse, sheep, cattle, pig, dog, cat, horse, and non-human primates, such as monkey, gorilla and chimpanzee. MSCs have the advantages of rich source, easy isolation, painless collection, and high legal and ethical acceptance. These traits make MSCs of intense therapeutic interest, as they represent a population of cells with the potential to treat a wide range of acute and degenerative diseases.

In the context of the invention, MSCs may be collected from various sources by the methods known in the art. For example, in the case of collecting bone marrow-derived MSCs, bone marrow can be obtained by needle from the iliac crest of a human or non-human animal subject with appropriate anesthetization, followed by density gradient centrifugation and selection of adherent cells. Alternatively, when collecting MSCs from dental pulp is desired, tissue specimens may be picked up from the gingiva of a human or non-human animal subject using a biopsy device and then subjected to collagenase digestion. In a preferred embodiment, the collection of MSCs may further comprise isolation of the MSCs from the cell culture by virtue of the differences in surface antigen markers. Non-limiting examples of the isolation methods include magnetic cell sorting (MACS), fluorescence activated cell sorting (FACS) and flow cytometry sorting (FCS).

The cell culture medium for culturing the MSCs can be any standard cell culture medium which provides adequate nutrition to the cells. Suitable culture media include but are not limited to Dulbecco's Modified Eagle's Medium (DMEM), alpha-minimum essential medium (α-MEM), Iscove's Modified Dulbecco's Medium (IMDM), Nutrient Mixture F-12 (Ham's F12), RPMI 1640, McCoy's 5A Medium, MesenPRO RS™ medium and a combination thereof, and other media formulations readily apparent to those skilled in the art. Such media can be easily prepared or obtained from commercial sources. Details of cell culture media and methods can be found in Methods For Preparation of Media, Supplements and Substrate For Serum-Free Animal Cell Culture Alan R. Liss, New York (1984) and Cell & Tissue Culture: Laboratory Procedures, John Wiley & Sons Ltd., Chichester, England 1996. The cell culture medium may be supplemented, with components, such as vitamins, proteins and sugars, growth factors, such as FGF and EGF, antibiotics, such as penicillin, streptomycin and tetracycline, fungicides, hormones, anti-oxidants and so on. If desired, blood fractions, such as fetal calf serum, human plasma and platelet-rich plasma (PRP), may be added to support the growth of cultured cells.

Generally, MSCs exhibit gradually reduced cell growth and eventually become senescent after several passages in culture, leading to a potential decrease in secretome contents. Therefore, in order to achieve the maximal paracrine effects, the primarily cultured MSCs are used to generate a conditioned medium between passage 1 and 10, preferably between passage 2 and 6. According to the invention, the MSCs are cultured and therefore conditioned in a serum-free basal culture medium by standard methods using aseptic processing and handling. The term “basal culture medium” as used herein may refer to any liquid culture medium containing inorganic salts, amino acids and vitamins usually required to support growth of mammalian cells not having particular nutritional requirements. In some preferred embodiments, the serum-free basal culture medium is growth factor-free. Examples of the basal culture medium include but are not limited to Basal Medium Eagles (BME), Minimum Essential Medium (MEM), Dulbecco's Modified Eagle's Medium (DMEM), Nutrient Mixture F-10 (HAM's F-10) and Nutrient Mixture F-12 (HAM's F-12). The serum-free basal culture medium can be supplemented with a serum replacement medium, such as that commercially available from Invitrogen-Gibco (Grand Island, N.Y., USA).

In one embodiment, the MSCs that reach 10%-90% confluence in the culture, preferably 30%-80% confluence in the culture, such as 50%-80% confluence in the culture, may be conditioned by culturing the cells in the serum-free basal culture medium. As the secretome in the serum-free basal culture medium, such as extracellular proteins, have reached a desirable level, the cell culture is harvested. In the preferred embodiments, the cell culture is harvested anytime between 3 hours and 120 hours of incubation or even more, preferably at the 3^{th}, 6^{th}, 12^{th}, 18^{th}, 24^{th}, 30^{th}, 36^{th}, 42^{nd}, 48^{th}, 60^{th}, 72^{nd}, 84^{th}, 96^{th}, 108^{th}, 120^{th} hour following the start of culturing in the serum-free basal culture medium, such as at the 72^{th} and 96^{th} hour following the start of the culturing and all hours therebetween. In another preferred embodiments, the MSC culture can be harvested once the cells are over 50% confluence, preferably between 70% to 100% confluence, such as 80-90% confluence.

According to the invention, the harvested cell culture is processed further to obtain an aqueous fraction with a molecular weight of no more than about 5 kDa. Such processing may be carried out by any conventional method capable of separating molecules based on molecular weight, examples of which may include gel filtration, density gradient purification, membrane filtration, ultrafiltration, centrifugation, ultracentrifugation and other like methods known in the art. In one embodiment, the cell culture may be first subjected to membrane filtration, centrifugation, or a combination thereof, to remove a major portion of cell debris and other insoluble substances to obtain a conditioned medium, followed by subjecting the conditioned medium to ultrafiltration through a filter membrane with a molecular weight cut-off of 5 kDa. In another embodiment, the cell culture is directly subjected to ultrafiltration through a membrane having a molecular weight cut-off of 5 kDa, examples of which include but are not limited to tangential flow filtration (TFF) with a molecular weight cut-off of 5 kDa. The □5 kDa fraction thus obtained, which exhibits the desired neuroprotective activity as stated below, may be subjected to additional purification procedures to remove unwanted substances, such as proteases and toxic chemicals. Methods of purification include gel chromatography, ion exchange chromatography, affinity chromatography, HPLC purification and the like.

MSCs have been shown to have potent therapeutic effects in a number of disorders involving neuronal death, such as traumatic brain injury (TBI), SAH, Alzheimer's disease and Huntington's Disease (Im W. S. and Kim M. H., supra; Ghonim H. T. et al., J.V.I.N., 2016 January, 8(5): 30-37; Martínez-Garza D. M. et al., supra; Turgeman G., supra; and Hasan A. et al., supra). As disclosed herein, the ≤5 kDa medium fraction obtained from MSC culture according to the invention is neuroprotective and enhances neuronal survival in vitro (as shown in Example 5 below). From the outcome measurements of the SAH rat model as shown in Example 4 and the D-gal-induced rat hepatic encephalopathy model as shown in Example 6, it can be appreciated that the intrathecal delivery of the 5 kDa medium fraction results in improved cerebral tissue oxygenation, decreased cerebral vasospasm and vasoconstriction, and reduced neuroinflammation in vivo, all of which are critical factors responsible for or contribute to neuronal damage in brain and found ameliorated. Herein, it is further demonstrated in Examples 7-9 that the 5 kDa medium fraction enhances the motility and also increases the neuronal activity in zebrafish and rat models. Taken together, the results demonstrated herein indicate the capability of the 5 kDa medium fraction for serving as a neuroprotective composition.

The term “neuroprotective” as used herein refers to a pharmaceutical composition which is capable of maintaining the survival and activity of neuronal cells, or maintaining or even recovering their neuronal functions, or relieving or alleviating one or more factors that may lead to neuronal damage (such as neuroinflammation, vasospasm, vasoconstriction, microvascular dysfunction and oxidative stress), even in pathological or harmful conditions. The term “neuroprotective” may encompass preventing the neuronal cells from being damaged in a subject and/or treating the neuronal damage after its emergence in the subject. In this regard, the term “preventing” includes reducing the severity/intensity of, or initiation of, the neuronal damage. The term “treating” or “treatment” includes alleviation of the neuronal damage after its emergence, amelioration of one or more of the symptoms caused by or leading to neuronal damage, or deceleration of the course of neuronal damage. In some embodiments, the term “treating” or “treatment” may refer to the reduction of neuron death in a subject suffering from a neurological disorder associated with neuron death, as compared to the rate of neuron death in a control subject having the same disorder but not receiving treatment or receiving a different treatment. Nevertheless, the term “neuroprotective” shall not be understood in the sense that there is always a 100% protection against the neuronal damage. As used herein, the term “neuronal damage” may refer to the damage that occurs to any cell type (e.g., neurons, astrocytes, glia cells) as a result of an illness or injury, which may in turn causes cell death or loss of cell function. The extent of neuronal damage can be determined by any method known in the art for visualizing neuronal function, such as electroencephalography, magnetic resonance imaging, computerized tomography, contrast angiography and Doppler ultrasonography.

In one aspect, the invention contemplates the medical use of the neuroprotective composition disclosed herein for treating a neurological disorder associated with neuronal damage in a subject, as well as a therapeutic method for treating a neurological disorder associated with neuronal damage in a subject, comprising administering to the subject an effective amount of the neuroprotective composition. As used herein, the term “neurological disorder associated with neuronal damage” may mean a neurological disease that is characterized by neuronal damage or has an aetiology that involves neuronal damage. In general, the medical use and the therapeutic method disclosed herein do not require that cell damage be detected in a subject prior to treatment, if the subject has a disorder known in the art to be associated with neuronal damage. Non-limiting examples of neurological disorders associated with neuronal damage include amyotrophic lateral sclerosis, Alzheimer's disease, Parkinson's disease, Huntington's disease, muscular dystrophy, multiple sclerosis, ischemic stroke, hemorrhagic stroke (such as primary subarachnoid hemorrhage (primary SAH), secondary SAH, traumatic SAH and intracerebral hemorrhage (ICH)), transient ischemic attack (TIA), and traumatic brain injury (TBI). A neurological disorder associated with neuronal damage can be diagnosed by a physician, a veterinarian or other clinician.

The data shown in the Examples below indicate that the neuroprotective composition disclosed herein is particularly effective in brain tissues to maintain the survival and activity of cerebral neuronal cells. In one preferred embodiment, the neurological disorder is associated with neuronal damage caused by neuroinflammation. It is known that brain hemorrhage, such as SAH, would lead to a cascade of neuro-inflammatory reactions, in which the receptor for advanced glycation endproducts (RAGE) associated intracellular signaling plays an important role for activation of mitogen-activated protein kinases (MAPKs) and nuclear factor κB (NF-κB). It has been reported that exogenous administration of recombinant sRAGE as a decoy to compete with the membrane-bound RAGE for ligand binding improved the outcome of mice I/R injury and further protected neurons from neuronal death (Wang K. C. et al., J. Cereb. Blood Flow Metab. 2017 Feb. 20; 37(2): 435-443), suggesting that attenuation or relief of neuroinflammation may be a useful measure in the treatment of brain hemorrhage. In addition, many neurological degenerative disorders have been proved to be associated with or caused by neuroinflammation, such as Alzheimer's disease, Parkinson's disease and Huntington's disease (McManus R. M. and Heneka M. T., Alzheimer's Research & Therapy 2017, 9:14, DOI 10.1186/s13195-017-0241-2; Gagne J. J. and Power M. C., Neurology 2010 Mar. 23, 74(12): 995-1002; and Im W. S. and Kim M. H., supra). In a preferred embodiment, the neurological disorder associated with neuronal damage is selected from the group consisting of Alzheimer's disease, Parkinson's disease, Huntington's disease, ischemic stroke, primary SAH, secondary SAH, traumatic SAH and intracerebral hemorrhage (ICH), transient ischemic attack (TIA), and traumatic brain injury (TBI). In a more preferred embodiment, the neurological disorder associated with neuronal damage is selected from the group consisting of Parkinson's disease, primary SAH and secondary SAH.

As used herein, the term “subject” is intended to encompass human or non-human vertebrates, such as non-human mammal. Non-human mammals include livestock animals, companion animals, laboratory animals, and non-human primates. Non-human subjects also include, without limitation, horses, cows, pigs, goats, dogs, cats, mice, rats, guinea pigs, gerbils, hamsters, mink, rabbits and fish. It is understood that the preferred subject is a human, especially a human patient afflicted with or at risk for a neurological disorder associated with neuronal damage, such as Parkinson's disease, primary SAH and secondary SAH.

For the purpose of research, the term “subject” may refer to a biological sample as defined herein, which includes but is not limited to a cell, tissue, or organ. Accordingly, the invention disclosed herein is intended to be applied in vivo as well as in vitro.

According to the invention, the term “administering” includes dispensing, delivering or applying the neuroprotective composition in a suitable pharmaceutical formulation to a subject by any suitable route for delivery of the neuroprotective composition, or a metabolome thereof, to the desired location in the subject to contact the neuroprotective composition, or a metabolome thereof, with target cells or tissues. In one embodiment, the neuroprotective composition is administered to a subject before, during and/or after an injurious event or the onset of a neurological disorder. In one embodiment, one or more therapeutic agents may be administered to a subject in conjunction with the neuroprotective composition. The neuroprotective composition can be administered prior to (e.g., 0.5 hours, 1 hour, 2 hours, 4 hours, 6 hours, 12 hours, 18 hours, 24 hours, 36 hours, 2 days, 3 days, 4 days, 5 days, 6 days, 7 days, or more), concurrently with, or after (e.g., 0.5 hours, 1 hour, 2 hours, 4 hours, 6 hours, 12 hours, 18 hours, 24 hours, 36 hours, 2 days, 3 days, 4 days, 5 days, 6 days, 7 days, or more) the administration of the one or more therapeutic agents. The neuroprotective composition and the therapeutic agent(s) can be administered by different regimens, e.g., different schedules, different routes of administration, or different doses.

The neuroprotective composition disclosed herein may be administered to the subject by any suitable route, such as a topical, enteral or parenteral route, for example, an oral, intravenous, intraarterial, subcutaneous, intramuscular, intraperitoneal, transdermal, transmucosal (such as nasal, sublingual, vaginal, buccal, rectal), intrathecal, intracranial or intracerebral route. Administration can be either rapid as by injection, or over a period of time as by slow infusion or administration of a slow release formulation.

In one preferred embodiment, the neuroprotective composition is to be administered intranasally and prepared in the form of an intranasally administrable formulation. Formulations for intranasal administration are known in the art and may be in the form of nasal droplets, a nasal spray or an aerosol formulation. The aerosol formulation may take the form of lyophilized powder, a suspension or a solution. In another preferred embodiment, the neuroprotective composition is to be administered via oral and throat mucosa and prepared in the form of a transmucosally administrable formulation.

In still another preferred embodiment, the neuroprotective composition disclosed herein is prepared as injectables, either as liquid solutions or suspensions, which are preferably isotonic with the blood of the recipient with a pharmaceutically acceptable carrier. Solid forms suitable for solution in, or suspension in, liquid prior to injection may alternatively be prepared. The injectable formulation may further include one or more pharmaceutically acceptable carrier. Suitable excipients may include, for example, water, saline, dextrose, glycerol, ethanol, wetting agents, emulsifying agents, and pH buffering agents. In some embodiments, the composition is in lyophilized form, in which case it may include a stabilizer, such as BSA. In some embodiments, it may be desirable to formulate the composition with a preservative, such as thiomersal or sodium azide, to facilitate long term storage. The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release, absorption or penetration across the blood-brain barrier.

The neuroprotective composition is administered to the subject in a therapeutically effective amount to elicit the biological or medicinal response that is being sought in a cell, tissue, system, animal or human by a researcher, veterinarian, medical doctor or other clinician and preferably to stabilize, ameliorate or alleviate one or more of the symptoms of the disease condition in the subject, such as stabilizing, ameliorating or alleviating neuroinflammation, vasospasm, vasoconstriction, hemiplegia, hyperflexia, weak muscles, twitching, speech problems, breathing problems, swallowing difficulties, loss of memory, confusion, disorientation, difficulty writing, depression, anxiety, social withdrawal, mood swings, aggressiveness, changes in sleeping habits, tremors, bradykinesia, rigid muscles, impaired balance, involuntary facial movements, numbness or weakness in limbs, partial or complete loss of vision, fatigue, dizziness, paralysis on one side of body or face, and headache. Therefore, the term “effective amount” is meant to refer to an amount of the neuroprotective composition which produces a medicinal effect observed as reduction in the symptoms above when the effective amount of the composition is administered to the subject. While the effective amounts are typically determined by the effect they have compared to the effect observed in the absence of the neuroprotective composition disclosed herein (i.e., a control), the actual dose is calculated dependent upon the particular route of administration selected. The actual dose may be calculated dependent upon the particular route of administration selected. Further refinement of the calculations necessary to determine the appropriate dosage for administration is routinely made by those of ordinary skill in the art. Thus, when administered to a human subject, the neuroprotective composition is preferably administered daily, weekly or twice a week, at an amount ranging from 0.01 mg/kg body weight/day to 100 mg/kg body weight/day, more preferably from 0.1 mg/kg/day to 10 mg/kg/day. Dose administration can be repeated depending upon the pharmacokinetic parameters of the dosage formulation and the route of administration used.

As disclosed herein, the invention further contemplates a medical use of the neuroprotective composition in inhibiting cerebral neuroinflammation in a subject in need thereof, as well as a therapeutic method of inhibiting cerebral neuroinflammation in a subject in need thereof. The term “neuroinflammation” as used herein may refer to the inflammatory reactions occurring in the nervous tissue, which may involve microglial activation, astrogliosis and release of numerous inflammatory mediators. The term “inhibit”, “inhibiting”, “suppress”, or “suppressing” used in the context of the invention means to decrease the amount, quality, or effect of a particular activity and refers to, for example, reduction in the severity of the inflammatory reactions occurring in the brain tissue as a result of administration of an effective amount of the neuroprotective composition to a subject, such as the results demonstrated in Examples 4 and 6.

The following examples are given for the purpose of illustration only and are not intended to limit the scope of the invention. It should be noted that, in the examples described below, the data of animal model were presented by grading. The clinical outcome was correlated with grading by logistic regression. The data of cell culture will be given by mean±SE. Cumulative concentration-response curves to different concentrations of CSF were obtained. Statistical analysis was performed by two-way ANOVA. Statistical significance is defined as p<0.05.

### Example 1: Isolation and Culture of Dental Pulp Stem Cells (DPSC)

After the upper tooth was totally harvested from a 3-week old male Wistar rat, the tissues were extracted using a syringe needle and were transferred into 25 cm² flask (Corning, Inc. NY, USA). The dental pulps were washed twice with phosphate buffered saline and treated with type II collagenase (50-100 U/mL) for an hour. The tissue was then centrifuged at 200 rpm for 4 minutes to obtain a cell mass, which was washed twice with phosphate buffered saline subsequently. Cells were cultured in Dulbecco's Modified Eagle Medium (DMEM) Nutrient Mix F12 (DMEM/F12; Life Technologies, Karlsruhe, Germany) supplemented with, 10% FBS (Gibco®, Grand Island, N.Y., USA) for several days, until the number of cells reached more than 1×10⁶. The cells were trypsinized to detach and washed twice with phosphate buffered saline and then resuspended in 0.5 mL phosphate buffered saline. The cell suspension was depleted of fibroblasts by incubating with anti-fibroblast antibody-conjugated magnetic beads at 4° C. for 15 minutes in a magnetic-activated cell separator. The eluate from the cell separator was centrifuged at 200 rpm for 4 minutes to obtain mesenchymal stem cells (MSCs). MSCs were detached by trypsin (Sigma Chemical Co., St. Louis, Mo., USA) when reached confluence and sub-cultured in 75 cm² flask (Corning, Inc. NY, USA) at the density of 2×10³ cells/cm², and then sub-cultured in 150 cm² flask (Corning, Inc. NY, USA) at the density of 5×10⁴ DPSCs at passage 3.

### Example 2: Preparation of Bioactive Medium Fraction

Conditioned medium was generated as follows: 80% confluent passage 2-5 MSCs in 150 cm² cell culture flask (Corning, Inc. NY, USA) were fed with, 20 mL/flask serum-free DMEM/F12 (Life Technologies, Karlsruhe, Germany) supplemented with, 10 ml Hank's Balanced Salt Solution (HBSS; Life Technologies, Carlsbad, Calif., USA) and 300 μl penicillin-streptomycin (1%), then incubated for 72 hours. For in vitro and in vivo experiments stated below, the conditioned medium was further concentrated using a tangential flow filtration (TFF) system (Millipore Co., St. Charles, Mo., USA) fitted with a 5 kDa cut-off membrane (Millipore Co., Billerica, Mass., USA) following the manufacturer's instructions, thereby obtaining a medium fraction of nominal molecular weight equal to and less than 5 kDa.

### Example 3: Rat SAH Model

Male Wistar rats (250 to 300 g) were used in the Example. All surgical procedures were performed according to the National Institutes of Health Guide for the Care and Use of Laboratory Animals, and animal experiments were approved by the local animal care and use committee. The anesthetic was 2.5% isoflurane with, 70% nitrous oxide and 27.5% oxygen. It was administered via a tracheostomy tube to ensure deep sedation as verified by an absence of hind limb and forelimb pain reflexes as well as the absence of corneal reflexes. Normal, non-labored breathing was maintained throughout the surgery. Temperature was monitored with a rectal probe, and body temperature was maintained at 37° C. with the animal on a thermal blanket. Blood pressure was monitored and maintained at 100-120 mmHg.

The animals were randomly divided into three groups, namely, the SAH group where SAH was induced in the rats by injecting 0.3 mL of fresh autologous blood into the cisterna magna over a 5-minute period with the animals being kept in a 20 degree head-down position, the Control group subjected to sham operation that received physiological saline rather than autologous blood, and the Treated group where the rats were administered with the medium fraction prepared in Example 2 intrathecally one hour before the SAH induction.

### Example 4: Microvasculature of Rat Cerebral Meninges After SAH

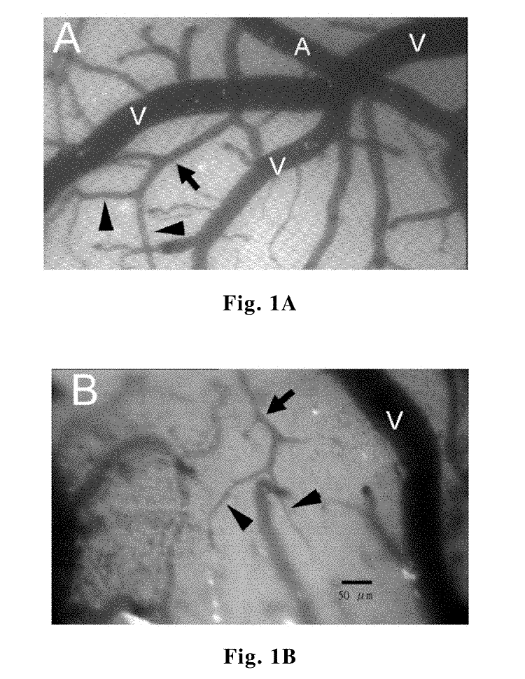

The microcirculation vasculature on the brain surface of the rat model was examined 24 hours after the SAH induction. A craniotomy of 5×5 mm was made behind the frontal suture of the rats. The dura was opened by a micro-scissor. A CAM1 laser doppler capillary anemometer (KK Technology, U.K.) having a high resolution (752×582 pixel) monochrome charge-coupled device (CCD) video camera was used to visualize the microcirculation vasculatures and measure the blood flow velocity in the cortical vessels. A dissection microscope is attached to a heavy support to allow three-dimensional adaptations without contact of brain surface. The field of vision was 684×437 μm and the image was magnified to give an overall magnification of about 0.91 μm/pixel. The results were shown in FIGS. 1A-1C. As shown in FIGS. 1A and 1B, where the arterioles and venules in rat brains were marked by the letters A and V, respectively, diffuse vasoconstriction of secondary arterioles (indicated by arrows) and terminal arterioles (indicated by arrowheads) was observed in the SAH group, in contrast to the Control group. However, the vasoconstriction was relieved in the Treated group, as shown in FIG. 1C.

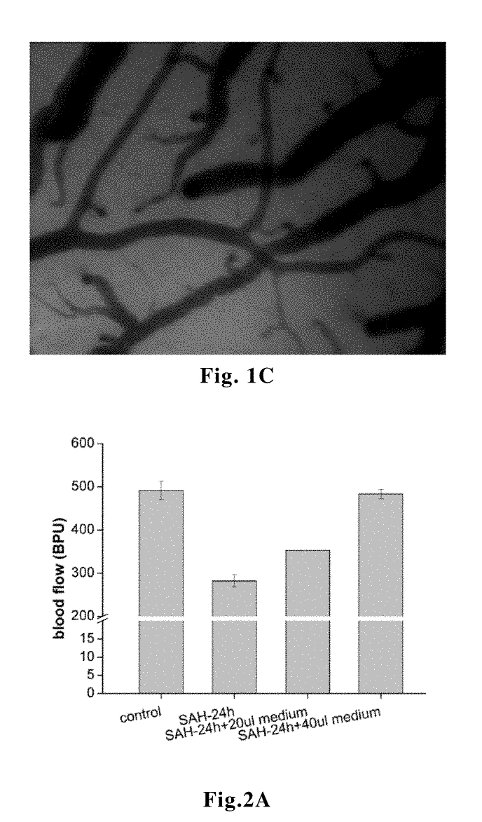

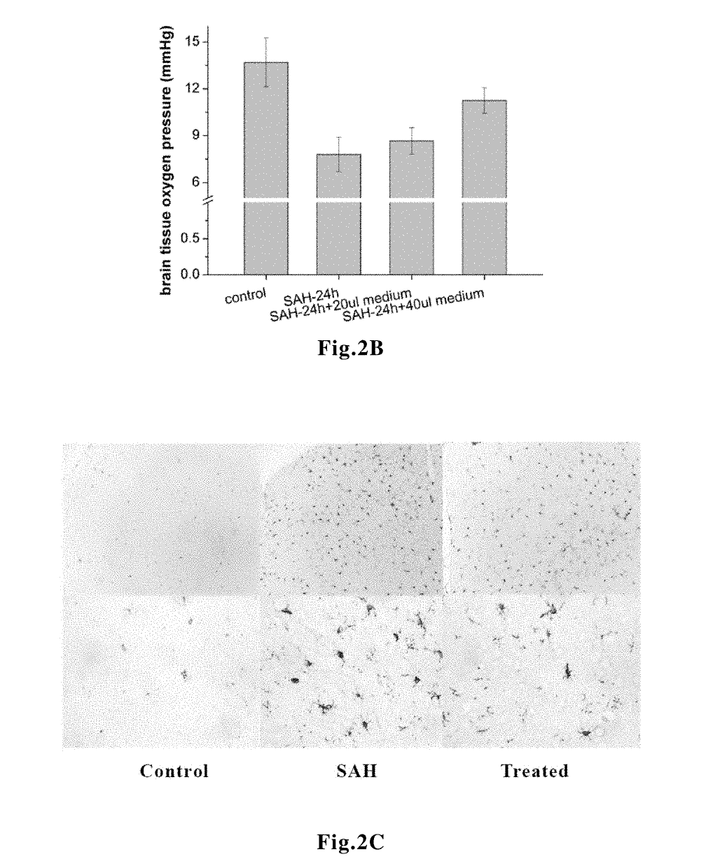

Microcirculation parameters, including blood flow and oxygen pressure, were further measured. Laser detectors (OxyLite 2000E and OxyFlow 2000E systems, Oxford Optronic Ltd., England) were employed to determine the blood flow and oxygen partial pressure in rat brains. As shown in FIGS. 2A and 2B, the regional blood flow and brain tissue oxygen pressure at the brain surface were significantly lower in SAH group as compared to the Control group at a depth of <4 mm from the brain surface, whereas dose-dependent increases in regional blood flow and brain tissue oxygen pressure were observed in the Treated group.

Cerebrospinal fluid (CSF) was also collected from the SAH group via cisterna magna at 24^{th} hour following the SAH induction.

The animals were sacrificed at 24^{th} hour post SAH induction, and their brains were fixed in 4% formaldehyde in PBS (freshly prepared from paraformaldehyde powder) overnight at 4° C. before being transferred to sequential 20% and 30% solutions of sucrose (w/v) at 4° C. until the brains sank to the bottom of the solution. The brains were embedded in a Tissue-Tek® Embedding Centre (Sakura, Torrance, Calif., USA) before sectioning (10 μm sections made using a cryostat) in the coronal anatomical plane. Sections were first exposed for a minimum of 30 min to PBS containing 0.1% Triton X-100 (Amresco, Santa Cruz, Calif., USA) and 10% normal goat serum (Sigma Chemical Co., St. Louis, Mo., USA) to block nonspecific antibody binding, followed by incubation with anti-Ibal antibody. Ionized calcium binding adaptor molecule 1 (Ibal) is a calcium-binding protein specifically expressed in microglial cells and its expression in microglia is up-regulated following neuroinflammation, nerve injury and central nervous system ischemia. FIG. 2C shows that the number of the Ibal-positive microglial cells was significantly lowered in the Treated group as compared to the SAH group, indicating less inflammation in the Treated group.

All of the data disclosed in this Example suggest that the intrathecal injection of the medium fraction prepared in Example 2 improved the cerebral tissue oxygenation and reduced cerebral vasospasm and inflammation and, therefore, provided neuronal protection in the animal model.

### Example 5: Neuronal Cell Viability Assay

It has been shown that Post-SAH cerebrospinal fluid from patients and rats induces neuronal cell death (Wang K. C. et al., supra). This Example was conducted to determine whether the reduced tissue damage observed in the Treated group in Example 4 was attributed to the direct beneficial effect of the medium fraction prepared in Example 2 on cortical neurons.

Dissociated cell neuron-enriched cultures of cerebral cortex were established from embryonic day 15 (E15) rat embryos. Cells were plated in 60-mm-diameter plastic or 35-mm glass-bottom dishes on a polyethyleneimine substrate in 0.8 ml of Minimum Essential Medium with Earle's salts supplemented with, 10% heat-inactivated FBS (Gibco®, Grand Island, N.Y., USA), 1 mM L-glutamine, 1 mM pyruvate, 20 mM KCl, 26 mM sodium bicarbonate (pH 7.2). Following cell attachment, the culture medium was replaced with Neurobasal Medium with B27 supplements (Gibco®, Grand Island, N.Y., USA). Experiments were performed in 7- to 9-day-old cultures. Approximately 95% of the cells in the cultures were neurons, and the rest of the cells were astrocytes. The cultured neurons were incubated with, 0.25 ml cerebrospinal fluid (CSF) collected from the SAH rats in Example 4 and 5 ml Locke's buffer. Control cultures were incubated in Locke's buffer containing 10 mM glucose.

Cell viability was evaluated with Alamar blue dye. Dissociated cells were counted and plated in 24-well plates and exposed to treatments for a pre-defined period. The culture medium was removed and replaced with 300 μl per well of 0.5% Alamar blue diluted in Locke's solution and incubated for 1-2 hours at 37° C. in a 5% CO₂ incubator. Levels of the Alamar blue reaction product were measured using a HTS 7000 Plus Bio Assay Reader (540-nm excitation and 590-nm emission wavelengths; purchased from Perkin-Elmer Inc., Wellesley, Mass., USA). Values for cultures exposed to experimental treatments were expressed as a percentage of the mean value for untreated control cultures.

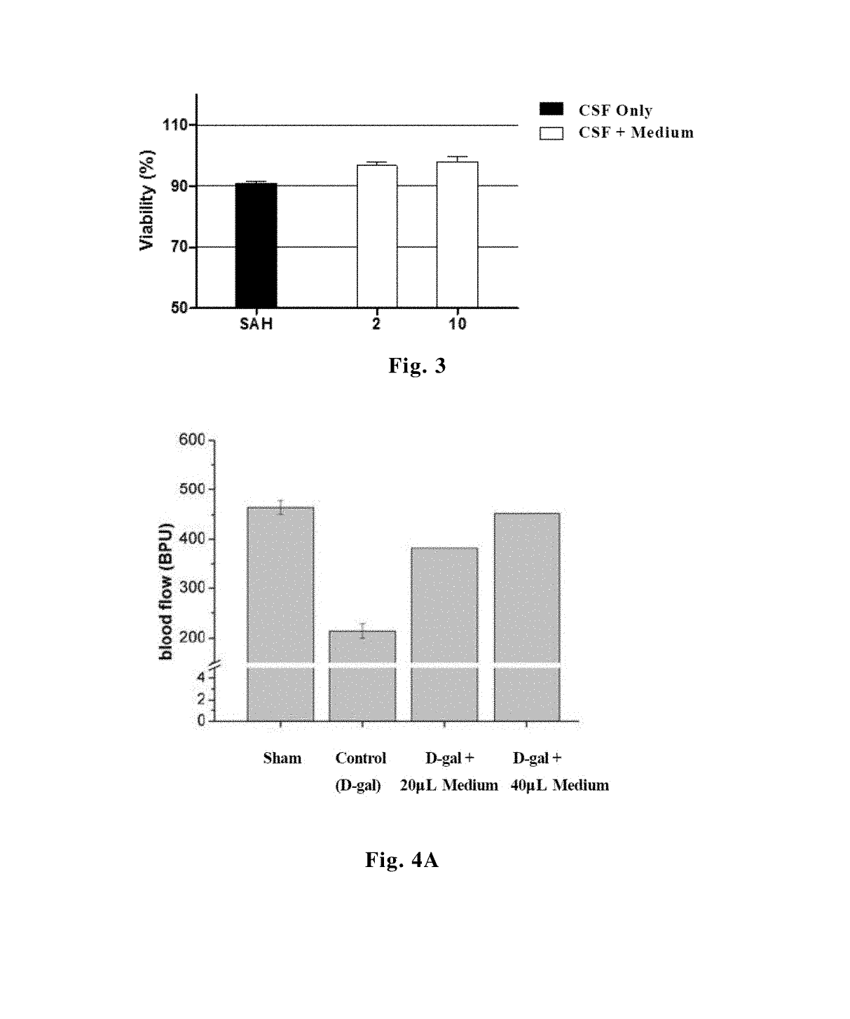

As shown in FIG. 3, the CSF collected from the rats in the SAH group induced neuronal death, while the exogenous administration of the medium fraction prepared in Example 2 (2 and 10 μg/ml) to the cultured neurons exposing to the CSF showed significantly less vulnerable to death. The results indicated that the medium fraction prepared in Example 2 exhibited protective effects against certain cytotoxic molecules inside the CSF retrieved from SAH patients.

### Example 6: D-gal Induced Hepatic Encephalopathy

Male Wistar rats (250 to 300 g) were randomly divided into three groups, namely, the Control group where rats received an intra-peritoneal injection of D-galactosamine (D-gal) (1000 mg/kg) once to induce acute hepatic failure, the Treated group where rats received an intrathecal injection of the medium fraction prepared in Example 2 three hours after the D-gal injection, and the Sham group where rats were neither treated with D-gal nor the medium fraction prepared in Example 2.

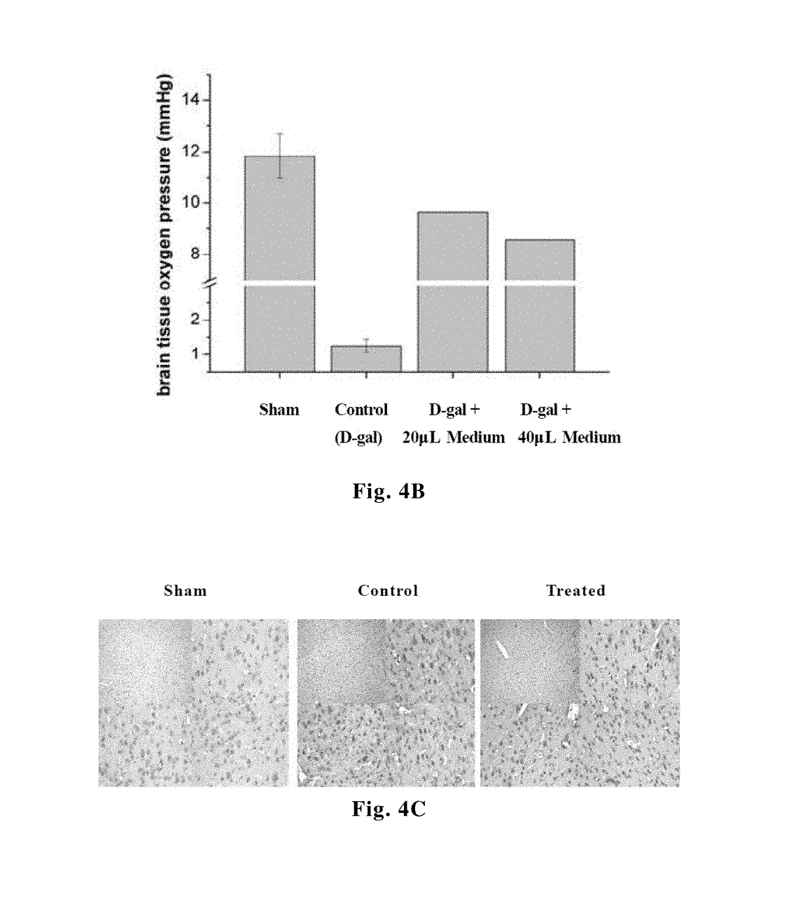

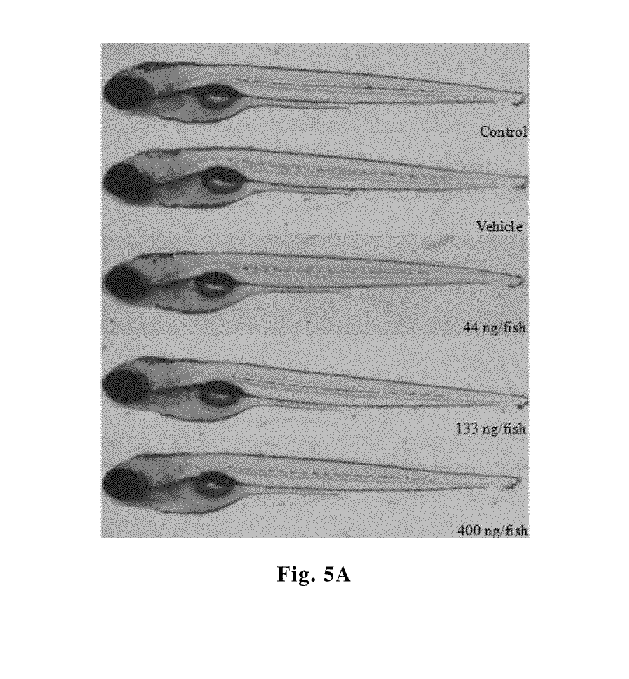

The microcirculation vasculature of rat cerebral meninges was observed and measured at 24^{th} hour after the D-gal injection according to the procedure stated in Example 4. As shown in FIGS. 4A and 4B, the regional blood flow and brain tissue oxygen pressure at the brain surface were significantly lower in the Control group as compared to the Sham group, whereas restoration in regional blood flow and brain tissue oxygen pressure were seen in the Treated group. The results indicate that the intrathecal injection of the medium fraction prepared in Example 2 reversed the cerebral microcirculation impairment caused by D-gal induced hepatic encephalopathy.

The animals were sacrificed at 48^{th} hour post D-gal injection, and their brains were fixed in 4% formaldehyde in PBS (freshly prepared from paraformaldehyde powder) overnight at 4° C. before being transferred to sequential 20% and 30% solutions of sucrose (w/v) at 4° C. until the brains sank to the bottom of the solution. The brains were embedded in a Tissue-Tek® Embedding Centre (Sakura, Torrance, Calif., USA) before sectioning (10 μm sections made using a cryostat) in the coronal anatomical plane. Sections were first exposed for a minimum of 30 min to PBS containing 0.1% Triton X-100 (Amresco, Santa Cruz, Calif., USA) and 10% normal goat serum (Sigma Chemical Co., St. Louis, Mo., USA) to block nonspecific antibody binding. Apoptosis of glia cells was assessed using the TUNEL Assay (Calbiochem/EMD Chemicals, Gibbstown, N.J., USA) according to manufacturer's instructions. FIG. 4C shows that the number of the TUNEL-positive glia cells was significantly lowered in the Treated group as compared to the Control group, suggesting that the injected medium fraction provided neuronal protection in the animal model.

### Example 7: Enhancement of Neuronal Activity

Thirty wild type AB zebrafish (Danio rerio) were transferred into a 6-well microplate. Zebrafish were treated with the medium fraction prepared in Example 2 (22, 67 and 200 ng/fish) by muscle injection, and the injection volume was 20 nL/fish. The medium fraction was serially diluted in physiological saline that served as a vehicle control. After treatment, 10 zebrafish from each group were monitored by an automated video tracking system for measuring the zebrafish total distance moved (S). Motility reduction was calculated using the following equation:

Motility Reduction (%)=(1−(S(sample)/S(Vehicle))×100%

The results are summarized in Table 1 below.

TABLE 1
Motility Reduction in Zebrafish (n = 10)

| | Zebrafish Total | |
|---|---|---|
| Group | Distance Moved | Motility Reduction Rate |
| (ng/fish) | (Mean ± SE) | (%) |
| Control | 7380 ± 501.5 | — |
| Vehicle | 7232 ± 586.5 | — |
| 22 | 6913 ± 611.6 | 4.4 |
| 67 | 7790 ± 418.6 | −7.7 |
| 200 | 8232 ± 493.5 | −13.8 |

The data shown in Table 1 above indicate that the treatment of the medium fraction prepared in Example 2 enhanced the motility and therefore the neuronal activity of zebrafish in a dose-dependent manner.

In a separate experiment, thirty wild type AB zebrafish larvae were transferred into a 6-well microplate. Zebrafish larvae were treated with the medium fraction prepared in Example 2 (44, 133 and 400 ng/fish) by yolk sac injection, the injection volume was 40 nL/fish. The medium fraction was diluted in physiological saline that served as a vehicle control. After treatment, zebrafish were stained with acridine orange, 10 zebrafish from each group were photographed under a fluorescence microscope to quantify zebrafish skin fluorescence intensity (S). Sample induced dermal toxicity was calculated using the following equation:

Skin Toxicity=[S(sample)/S(Vehicle)−1]×100%

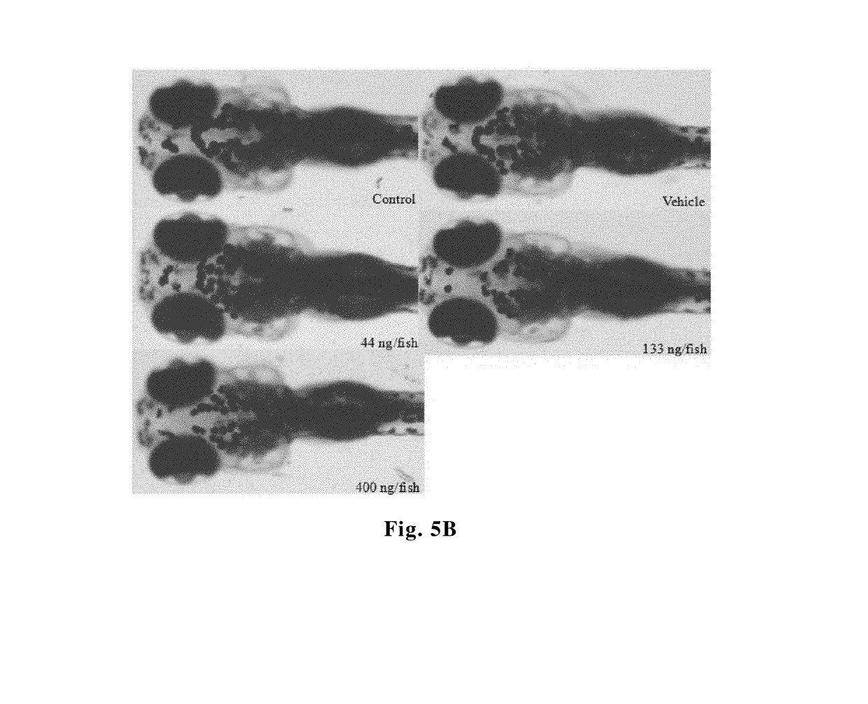

The results are shown in FIGS. 5A and 5B and further listed in Table 2 below.

TABLE 2
Skin Toxicity in Zebrafish (n = 10)

| Group | Fluorescence Units | Skin Toxicity |
|---|---|---|
| (ng/fish) | (Mean ± SE) | (Apoptosis rate) |
| Control | 387540 ± 8253 | — |
| Vehicle | 378039 ± 7927 | 0 |
| 44 | 396228 ± 10229 | 4.8% |
| 133 | 364410 ± 11167 | −4.0% |
| 400 | 385423 ± 13837 | 2.0% |

Table 2 indicates that the medium fraction prepared in Example 2 was not toxic to zebrafish skin. FIGS. 5A and 5B show that neither abnormal skin and muscle pattern alternation, nor abnormal pigmentation, was observed in zebrafish treated with the medium fraction prepared in Example 2.

### Example 8: Anti-Parkinson's Disease Efficacy Test

Thirty wild type AB zebrafish larvae were transferred to 6-well microplates. Zebrafish were treated with the neurotoxin 6-hydroxydopamine (6-OHDA) to induce Parkinson's Disease model. Nomifensine, a norepinephrine-dopamine reuptake inhibitor commercially available in the market, was employed as the positive control drug, delivered by soaking at a final concentration of 1.5 μg/mL. The medium fraction prepared in Example 2 was tested at 44, 133 and 400 ng/fish, delivered by yolk sac injection. Both nomifensine and the medium fraction were co-treated with 6-OHDA. After treatment, fish larvae were transferred from the 6-well plates to a 96-well plate, one fish in each well with, 200 μL solution, then the plate was loaded into a zebrafish-specific behavior analyzing system (Viewpoint, France), and the motility of each larva was recorded for 30 minutes, 10 larvae for each group. The total swim distance (D, in mm) of larva was measured as the endpoint to assess the efficacy of Parkinson's disease treatment. The anti-Parkinson's Disease efficacy was calculated using the following equation:

Efficacy={[D(sample)−D(model)]/D(control)−D(model)]}×100%

The results are shown in FIGS. 5A and 5B and further listed in Table 3 below.

TABLE 3
Anti-Parkinson's Disease Efficacy in Zebrafish Model (n = 10)

| | | Anti-Parkinson's |
|---|---|---|
| Group | Total Distance(mm) | Disease Efficacy |
| (ng/fish) | (Mean ± SE) | (Motility Increase Rate) |
| Control | 4366 ± 373*** | — |
| Model (6-OHDA) | 1611 ± 270 | — |
| Nomifensine | 2738 ± 165* | 40.9%* |
| 44 | 2686 ± 202* | 39.0%* |
| 133 | 3178 ± 311*** | 56.9%*** |
| 400 | 2652 ± 160* | 37.8%* |

Compared with Model,
*p < 0.05,
***p < 0.001.

In the 6-OHDA induced Parkinson's Disease-like Zebrafish Larvae Model assays, the Parkinson's disease-like movement disorder was significantly rescued by the medium fraction prepared in Example 2 at the three tested concentrations, indicating that the medium fraction exhibited neuroprotective effects on zebrafish embryos exposed to 6-OHDA.

### Example 9: Parkinson's Disease Rat Model

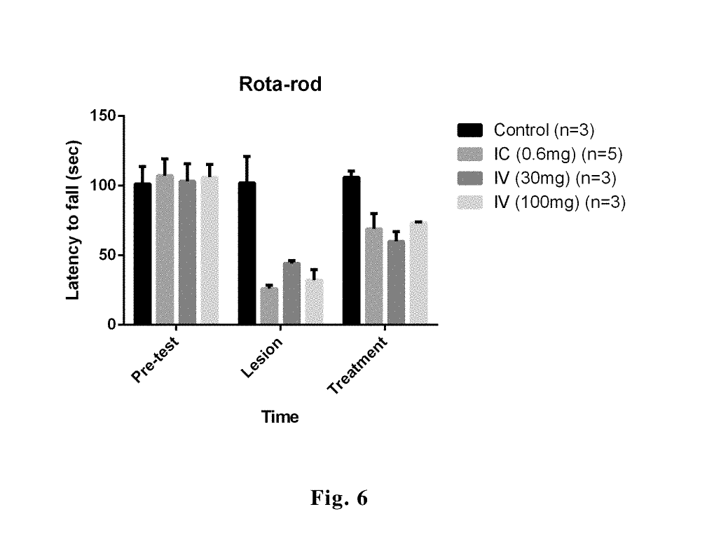

Male Lewis rats (8 weeks old) were randomly divided into four group. Rats in the Control group were injected intraperitoneally with dimethyl sulfoxide daily, and rats in the three Treatment groups were injected intraperitoneally for two weeks with rotenone (2 mg/kg/day) dissolved in dimethyl sulfoxide. Motor coordination of the animals in the respective groups was assessed before and after rotenone treatment using a rotarod apparatus equipped with a rotating rod of 3.1 cm in diameter (Ugo Basile model 7700, Veresi, Italy). In the rotarod test, animals were first exposed to a 3-day prior training session to acclimatize them on rotarod before the actual assessment on day 4. The animal's average latency to fall from the rotarod shown in FIG. 6 indicated that the rotenone-lesioned rats revealed typical Parkinson's Disease symptoms and therefore had significantly lower performance compared to the Control. After the rotenone treatment, rats in the Treatment groups received daily intracranial injection (IC) of 0.6 mg of the medium fraction prepared in Example 2 for two weeks, or daily intervenus injection (IV) of 30 mg of the medium fraction prepared in Example 2 for two weeks, or daily intervenus injection of 100 mg of the medium fraction prepared in Example 2 for two weeks. The rotarod test was performed again in the respective groups, and the animal's average latency to fall from the rotarod was recorded. As shown in FIG. 6, the administration of the medium fraction disclosed herein ameliorated the rotenone-induced motor coordination impairment in the rat model.

While the invention has been described with reference to the preferred embodiments above, it should be recognized that the preferred embodiments are given for the purpose of illustration only and are not intended to limit the scope of the present invention and that various modifications and changes, which will be apparent to those skilled in the relevant art, may be made without departing from the spirit and scope of the invention.

All papers, publications, literature, patents, patent applications, websites, and other printed or electronic documents referred herein, including but not limited to the references listed below, are incorporated by reference in their entirety. In case of conflict, the present description, including definitions, will prevail.

What is claimed is:

1. A method for treating Parkinson's disease in a subject, comprising administering to said subject an effective amount of a neuroprotective composition to suppress neuronal damage; wherein the neuroprotective composition is obtainable by a process comprising the steps of:
(i) culturing mesenchymal stem cells in a serum-free basal culture medium for at least 3 hours to obtain a cell culture; and
(ii) processing the cell culture obtained in step (i) to obtain an aqueous fraction with a molecular weight of no more than about 5 kDa as the neuroprotective composition.

2. The method according to claim 1, wherein the mesenchymal stem cells are dental pulp mesenchymal stem cells.

3. The method according to claim 2, wherein the processing step (ii) comprises ultrafiltrating the cell culture obtained in step (i) through a membrane having a molecular weight cut-off of 5 kDa, thereby collecting a filtrate passing through the membrane.

4. The method according to claim 3, wherein the subject is selected from the group consisting of human and non-human vertebrates.
